SEQUENCE LISTING (12 sequences)

SEQ ID NO: 1 - DNA, 42 bp, Artificial Sequence
  Forward primer
ccgccggaattccatatgcactacaagcagcactgccctcca

SEQ ID NO: 2 - DNA, 51 bp, Artificial Sequence
  Reverse primer
ccgccggaattctttatcatcatcatcggatccgactggctcccagcagtc

SEQ ID NO: 3 - DNA, 45 bp, Artificial Sequence
  forward primer
ccgccgggatccgatgatgatgataaaacgccattaggcccggcc

SEQ ID NO: 4 - DNA, 39 bp, Artificial Sequence
  Reverse primer
ccgccggaattcaagccttaacggctccgctaaatgacg

SEQ ID NO: 5 - DNA, 138 bp, Artificial Sequence
  GM tag nucleotide sequence
atgcactacaagcagcactgccctccaaccccggaaacttcctgtgcaacccagattatcacctttgaaagtttcaaagagaacctgaaggactttctgcttgtcatcccctttgactgctgggagccagtcggatcc

SEQ ID NO: 6 - protein, 46 aa, Artificial Sequence
  GM tag amino acid sequence
MHYKQHCPPTPETSCATQIITFESFKENLKDFLLVIPFDCWEPVGS

SEQ ID NO: 7 - DNA, 321 bp, Artificial Sequence
  SD nucleotide sequence
atgagcgattccgattcagactcggactcggattccgattccgacagtgattcagattctgactcagattccgattctgattctgattcggattccgactccgatagcgactcagatagtgactctgactcggacagcgattctgatagcgactctgattccgatagcgatagcgattcagatagcgattctgactcggattctgattccgattctgactctgacagcgattccgatagcgacagcgactctgatagtgattcagactctgattctgatagtgatagcgattcggatagtggatccgatgatgatgataaa

SEQ ID NO: 8 - protein, 107 aa, Artificial Sequence
  SD amino acid sequence
MSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSGSDDDDK

SEQ ID NO: 9 - DNA, 531 bp, Artificial Sequence
  Human GCHF nucleotide sequence
atgacgccattaggcccggcctctagcctgccacagagcttcttactgaagtgcttagaacaagtgcgcaagatccaaggcgatggtgccgccttacaggaaaagctgtgcgccacctacaagttatgccatccggaggagctggtgctgttaggtcactctttaggcattccatgggccccactgtcttcttgcccaagccaggccctgcaactggccggctgtctgtctcagctgcattctggcctgtttctgtatcagggcttactgcaagcgctggagggcatcagcccagaattaggtccaaccctggacaccttacaactggatgtggccgatttcgcgacgaccatttggcagcagatggaagagctgggtatggccccagcgttacagccaacgcagggtgccatgccggcctttgcgagcgcctttcaacgtcgtgcgggtggtgttctggttgccagccacctgcaatcttttttagaggtgagctaccgcgtgttacgtcatttagcgcagccgtaatga

SEQ ID NO: 10 - protein, 175 aa, Artificial Sequence
  Human GCHF amino acid sequence
MTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 11 - DNA, 177 bp, Artificial Sequence
  His-GM tag nucleotide sequence
atgcatcatcatcatcatcatcatcactacaagcagcactgccctccaaccccggaaacttcctgtgcaacccagattatcacctttgaaagtttcaaagagaacctgaaggactttctgcttgtcatcccctttgactgctgggagccagtccaggaggatgatgatgattacgta

SEQ ID NO: 12 - protein, 59 aa, Artificial Sequence [sequence omitted]

(19) United States
(12) Patent Application Publication
(10) Pub. No.: US 2012/0107876 A1
(43) Pub. Date: May 3, 2012

(54) NOVEL FUSION TAG OFFERING SOLUBILITY TO INSOLUBLE RECOMBINANT PROTEIN

(75) Inventors: Sampali Banerjee, Maharashtra (IN); Sriram Padmanabhan, Maharashtra (IN)

(73) Assignee: LUPIN LIMITED, Mumbai (IN)

(21) Appl. No.: 13/318,276

(22) PCT Filed: Apr. 29, 2010
(86) PCT No.: PCT/IN10/00279

§ 371 Date: Jan. 11, 2012

(30) Foreign Application Priority Data
May 1, 2009 (IN) 689/KOL/2009

(51) Int. Cl. C12P 21/02; C12N 15/70; C07K 14/00

(52) U.S. Cl. 435/69.7; 530/350; 435/320.1

(57) ABSTRACT

The invention relates to a fusion tag comprising Serine-aspartic acid repeats of the well conserved region of the Staphylococcus aureus Sdr C gene superfamily. A START codon and an enterokinase cleavage site has been incorporated into this repeat region to make a novel fusion tag that is responsible for expressing soluble proteins in bacterial system.

### FIELD OF INVENTION

The invention relates to a fusion tag comprising Serine-aspartic acid repeats of the well conserved region of the Staphylococcus aureus Sdr C gene superfamily. A START codon and an enterokinase cleavage site has been incorporated into this repeat region to make a novel fusion tag that is responsible for expressing soluble proteins in bacterial system. The invention also involves a kit for expression of soluble proteins. The present invention also relates to a method of improving the solubility of protein when the protein is produced in vivo.

### BACKGROUND OF THE INVENTION

The advent of recombinant DNA technology and its application has made a number of recombinant therapeutics available for human use. Prokaryotic or eukaryotic (yeast and mammalian) expression systems are generally used for recombinant protein production. Among these, E. coli has been widely used for recombinant protein production. The system offers high productivity, high growth and production rate, ease of use and economy. E. coli facilitates protein expression by its relative simplicity, is inexpensive, fast growth, well-known genetics and the large number of compatible tools available for biotechnology. Especially the varieties of available plasmids, recombinant fusion partners and mutant strains have also advanced the possibilities of obtaining recombinant therapeutics with E. coli system. However, there are a few disadvantages as lack of post translational modifications, lack of proper secretion system for efficient release of produced protein into the growth medium, inefficient cleavage of amino terminus methionine which can result in lower protein stability increased immunogenicity, limited ability to facilitate extensive disulphide bond formation, improper folding resulting in inclusion body formation.

Inclusion bodies produced in E. coli are composed of densely packed denatured protein molecules in the form of particles and proteins residing in inclusion bodies are often inactive. In order to get an active protein, optimization of the expression conditions or the refolding studies are required which could be time consuming and cost intensive. On the other hand, many mammalian proteins can not be expressed successfully in E. coli which leaves researchers either to explore expression in a wide range of organisms like baculovirus expression system, gram positive organisms, Pseudomonas expression system and E. coli hosts at different temperatures along with various fusion tags.

Since insoluble proteins expressed in E. coli hosts require, complicated in vitro renaturation step and is indeed a low efficient process and even complex for proteins with multiple disulphide bonds, there is always a necessity for production of soluble recombinant protein in E. coli as the purification of highly expressed soluble protein is less expensive and time consuming than refolding and purification from inclusion bodies. Soluble protein production in E. coli is still a major bottleneck for researcher and many attempts have been undertaken to improve the solubility or folding of recombinant protein produced in E. coli. Of various strategies, co-expression of chaperone proteins such as E. coli GroEs, GroEl, DnaK and DnaJ, lowering incubation temperature, use of weak promoters, addition of sucrose and betaine in growth medium, use of richer medium with phosphate buffer such as TB, translocation to periplasm, fermentation at extreme pH, and use of fusion tags are examples of a few approaches.

Also, proteolytic degradation of recombinant proteins represents a major problem related to production of gene products in heterologous hosts. Several alternative strategies for stabilization of expressed gene products are available many of which often give dramatic stabilization effects. Optimization of fermentation conditions or downstream processing schemes together with these strategies is solutions to these problems. Various genetic approaches to improve the stability of recombinant proteins include (i) choice of host cell strain, (ii) product localization, (iii) use of gene fusion partners, and (iv) product engineering. In addition, the solubility of the gene product can be influenced by factors such as growth temperature, promoter strength, fusion partners, and site-directed changes. Altogether, a battery of approaches can be used to obtain stable gene products.

One of the best approaches to deal with solubility and stability has been to express proteins as N- or C-terminus fusions. Prior art show that formation of secondary structures in transcribed mRNA reduces expression of heterologous genes. These secondary structures interfere with the binding of ribosome with mRNA thereby prevent efficient translation initiation. These deleterious secondary structures more likely occur due to short-range RNA-RNA interactions. Sequence determinants at both N- and C-termini of proteins can influence their stability towards protease degradation. Although various alterations of expression conditions can sometimes solve the problem, the best available tools to date have been fusion tags that enhance the solubility of expressed proteins. However, a utility of these solubility fusions has been difficult since many proteins react differently to the presence of different solubility tags with some tags resulting in incorrect folding and some causing inactivity of some proteins.

Proteins do not naturally lend themselves to high-throughput analysis because of their diverse physiochemical properties. Consequently, affinity tags have become indispensable tools for structural and functional proteomics initiatives. Affinity tags are highly efficient tools for protein purification. They allow the purification of virtually any protein without any requirement of any prior knowledge of its biochemical properties. Though originally developed to facilitate the detection and purification of recombinant proteins, in recent years the fusion tag has become clear that affinity tags can have a positive impact on the yield, solubility and even the folding of their fusion partners. However, no single affinity tag is optimal with respect to all of these parameters; each has its strengths and weaknesses. Therefore, combinatorial tagging might be the only way to harness the full potential of affinity tags in a high-throughput setting.

There are several fusion tags available for the ease of expression and purification of recombinant proteins and the smallest fusion tag available is His-tag (6-10 aa). This has potential problems of leakage of Ni²⁺ ions used during for purification of His-tag proteins. The other tags available are thioredoxin (109aa), Glutathione S-transferase (236aa), maltose binding protein (363aa), NusA (435 aa) etc. Most of these tags are affinity tags are large in size and mostly they facilitate purification of the fused protein. Some of them are (thioredoxin, NusA etc) also reported to increase the solubility of the target proteins compared to unfused proteins when over expressed. Therefore, all the above-mentioned fusion tags are either affinity tags or they offer solubility. The advent of high-throughput structural genomics programs and advances in cloning and expression technology afford us a new way to compare the effectiveness of solubility tags and the use of affinity tags has therefore become widespread in several areas of research e.g., high throughput expression studies aimed at finding a biological function to large numbers of yet uncharacterized proteins.

US2006/0234222 discloses method of producing a soluble bioactive domain of a protein, the method comprising the step of selecting suitable soluble subunits of a protein and assessing the produced protein for desired activity. The method may comprise the steps of amplifying DNA encoding at least one candidate soluble domain, cloning the amplified DNA into at least one expression vector, using each of said vectors into which the DNA has been cloned to each transfect or transform one or more host cell strains, expressing said DNA in one or more host cell strains, and analyzing expression products from said host cells for solubility.

U.S. Pat. No. 6,861,403 discloses method for expressing proteins as a fusion chimera with a domain of p26 or alpha crystalline type proteins to improve the protein stability and solubility when over expressed in bacteria such as E. coli is provided. Genes of interest are cloned into the multiple cloning site of the Vector System just downstream of the p26 or alpha crystalline type protein and a thrombin cleavage site. Protein expression is driven by a strong bacterial promoter (Tac). The expression is induced by the addition of 1 mM IPTG that overcomes the lac repression (lac L_{c}). The soluble recombinant protein is purified using a fusion tag.

U.S. Pat. No. 6,613,548 relates to fusion products prepared by recombinant DNA procedures. The products are comprised of a soluble protein of interest and an insoluble proteinaceous tag.

Thus it is known that protein solubility is one of the major problems associated with over expressing proteins in bacterial system. Protein solubility is judged empirically by assaying the levels of recombinant protein in the supernatant and pellet of lysed cell extract. In general proteins with more hydrophilic residues can be found in soluble fractions of bacterial extracts. In contrast proteins rich in hydrophobic residues or proteins having complex secondary or tertiary structures are typically insoluble and are found in inclusion bodies. While in the form of inclusion bodies, the protein will have no biological activity and will be impossible to purify using affinity fusion tags. These inclusion bodies can be re-solubilised in chaotropic buffers such as 8M urea or 6M guanidine hydrochloride, but then must be slowly dialyzed against physiological buffers in an effort to refold and regain biological function. Due to the individual characteristics of each protein, this is a slow and painstaking process that may never produce active or useful protein. Therefore, the ability to quickly produce and screen soluble protein in bacteria such as E. coli represents a major step forward in protein biochemistry.

Thus the present invention aims at solving the problems of insoluble protein production by using a fusion tag, the fusion tag comprising Serine-aspartic acid repeat region of Staphylococcus aureus SdrC gene superfamily with a START codon and an enterokinase cleavage site to improve the solubility of those proteins which express as insoluble proteins. Further presence of affinity tags with this fusion tag of present invention would provide ease of purification.

### Objectives of the Present Invention

The object of the present invention is a fusion tag comprising Serine-aspartic acid repeat region of Staphylococcus aureus SdrC gene superfamily with a START codon and an enterokinase cleavage site.

Another object of the present invention is the use of fusion tag comprising Serine-aspartic acid repeat region of Staphylococcus aureus SdrC gene superfamily with a START codon and an enterokinase cleavage site to increase the solubility of proteins.

Another object of the present invention is a vector comprising fusion tag comprising Serine-aspartic acid repeat region of Staphylococcus aureus SdrC gene superfamily with a START codon and an enterokinase cleavage site and additional amino acids at the N terminal region of the serine aspartic acid repeat units

Another object of the present invention is a kit for expression of soluble proteins comprising vector comprising a Fusion tag comprising of additional aminoacids at the N terminal region and the SD repeat region of Staphylococcus aureus SdrC gene superfamily with a START codon and an enterokinase cleavage site actually offers the solubility factor for the gene of interest.

Another object of the present invention involves a method for producing soluble and active recombinant protein comprising: (a) cloning fusion tag comprising SD repeats in the vector (b) cloning additional amino acid sequence in step (a) (c) introduction of gene of interest in step (b) (d) Transformation of vector from step (d) in E Coli (e) expression of fusion protein (f) Separation of protein of interest from fusion protein.

### BRIEF DESCRIPTION OF THE ACCOMPANYING DRAWINGS

FIG. 1: Colony PCR with T7 forward and GM reverse primers.

FIG. 2: XbaI/SnaBI digestion of the pCGMSD construct

FIG. 3: Colony PCR with T7 reverse and forward primer

FIG. 4: Clone map for GMSD-GCSF

FIG. 5: SDS-PAGE profile of expressed GMSD-hGCSF

FIG. 6: Clone map for GMSD-hIL11

FIG. 7: Clone map for GMSD-hIL2

FIG. 8: Clone map for GMSD-Reteplase

FIG. 9: SDS-PAGE profile of expressed GMSD-hIL11

FIG. 10: SDS-PAGE profile of expressed GMSD-hIL2, enterokinase and Reteplase

### DETAILED DESCRIPTION OF THE INVENTION

The present invention provides a method for improving the solubility of target protein when the target protein is produced in bacteria

Another embodiment of the present invention provides a method for expressing target protein using a vector comprising a fusion tag, comprising serine-aspartic acid (SD) repeat region of SdrC protein family along with gene of interest with additional amino acids, about 10 to about 300amino acids at the N terminal region. The additional amino acids may be either derived from vector sequences from MCS or the sequences could be from extraneous polypeptides that aid in hyper expression of proteins. The additional amino acids could be used for affinity purification, antibody detection also. The vector when introduced in E. coli would express soluble proteins.

As used herein, the term “tagging” refers to introducing by recombinant methods one or more nucleotide sequences encoding a peptide tag into a polypeptide encoding gene. “Fusion protein” refers to the protein whose N terminus is formed by the fusion tag comprising the C terminus portion of human GM CSF and a non GM peptide at the C terminus.

The fusion protein must be continuous with the target protein. The same open reading frame of the target protein must be maintained with respect to the open reading frame of the fusion tag. Stop codons between the target protein and the fusion partner must be omitted.

Vectors suitable to be used for the present invention are numerous and a list of the vectors can be found in the art. The vectors commercially available from Stratagene, Promega, CLONTECH, Invitrogen GIBCO Life Sciences and other companies making expression vectors. All the vectors with bacterial promoters may be used.

Vectors particularly suitable are plasmid vectors, which include prokaryotic, eukaryotic and viral sequences. A list of these vectors can be found in Gene Transfer and Gene Expression: A Laboratory Manual, Ed. Kriegler, M., Stockton Press, New York (1990) and Molecular Cloning, A Laboratory Manual, CSH Laboratory Press, Cold Spring Harbor, N.Y. and Current Protocols in Molecular Biology, Vol. 1, Supplement 29, section 9.66, Ed. Asubel, F. M. et al., John Wiley & Sons (2001).

The present invention involves a fusion tag comprising the serine-aspartic acid repeat (SD) region of SdrC protein family of a gram positive bacterium, Staphylococcus aureus.

Another embodiment of the present invention involves a fusion tag comprising serine-aspartic acid repeat (SD) region of SdrC protein family of a gram positive bacterium, Staphylococcus aureus which comprises of 55 each of serine and aspartate residues along with additional amino acids, about 10 to about 300amino acids at the N terminal region. The additional amino acids may be either derived from vector sequences from MCS or the sequences could be from extraneous polypeptides that aid in hyper expression of proteins. The additional amino acids could be used for affinity purification, antibody detection also. The additional amino acid sequence may be any which is known in the art such as GST tag, His tag, T7 tag Trx tag, MBP tag, His-GM tag etc.

The most preferable is a 45 amino-acid long peptide and is the C-terminus part of human Granulocyte Macrophage Colony Stimulating Factor (hGMCSF) gene product. hGMCSF is a glycoprotein growth factor that induces proliferation of hematopoetic proginator. The processed hGMCSF polypeptide is 127 amino acid long and of molecular mass of 14.36 kDa. This tag is small and hence upon expression, the molar ratio of the gene of interest would be highest for a tag which is the smallest in size since the other well known tags are very large in size.

His-GM tag was prepared by modifying the GM tag by incorporating six histidine amino acids at the N-terminus of the GM tag.

There are three members of the cell surface-associated serine-aspartate family of proteins in S. epidermidis, namely, SdrF, SdrG (Fbe), and SdrH, and they are all characterized by the distinctive serine-aspartate dipeptide (SD) repeats. The overall structure of the coding region was found to follow the general pattern observed in other Sdr family proteins and included a signal sequence, an A domain, a repetitive domain termed BX, an SD repeat region, a cell wall anchor region with an LPXTG motif sequence (LPDTG, amino acids 674 to 678), a hydrophobic membrane-spanning region, and a series of positively charged residues at the C terminus.

Serine-aspartate repeats have previously been shown to allow a high degree of discrimination in S. aureus. Initial surveys revealed the largest amount of size variation in sdrG PCR amplicons, and the gene was present in all strains surveyed.

There were three differently sized PCR amplicons of the SD repeat region from the 48 strains analyzed (˜200 bp, ˜4 to 500 bp, and ˜8 to 900 bp), and there was 100% concordance between the size of the PCR fragment and the number of repeat cassettes.

The DNA sequence revealed 69 alleles of the repeat cassette, composed of 1 21-bp, 4 12-bp, and 64 different 18-bp repeats. The SD repeats had earlier been found in the S. aureus fibrinogen-binding clumping factors ClfA and ClfB. The clfA and clfB genes encode high-molecular-mass fibrinogen-binding proteins that are anchored to the cell surface of S. aureus.

SdrC family of proteins are membrane bound protein and consists of several functional domains. The C termini contain LPXTG motifs and hydrophobic amino acid segments characteristic of surface proteins covalently anchored to peptidoglycan. The fibrinogen-binding clumping factor protein of S. aureus is distinguished by the presence of a serine-aspartate (SD) dipeptide-repeat region. These Sd repeats span the cell wall and extend the ligand binding region from the surface of the bacteria and sdrC gene is abundant as a surface protein in several staphylococcus strains. Thus these SD-repeat regions would most probably enhance the solubility and promote the proper folding of its fusion partners in E. coli. Also, both the serine and aspartic acid are polar amino acids and has a high solubility offering solubility of otherwise insoluble proteins.

One of the embodiments of the present invention involves the method of producing soluble protein the method comprising (a) cloning of SD repeats in the vector (b) cloning of additional aminoacids in the N terminal region of SD repeat units in step (a) (c) introduction of gene of interest in step (b) (d) Transformation of vector from step (d) in E. Coli (e) expression of fusion protein (f) Separation of protein of interest from fusion protein.

The present invention also involves a kit comprising a vector comprising a fusion tag comprising Serine aspartic acid repeat units. The kit may be used for providing soluble and active protein of interest.

### DESCRIPTION OF A PREFERRED EMBODIMENT OF THE PRESENT INVENTION

### Example 1

### Construction of Fusion Tag Vector

The serine-aspartate (SD) repeat region was synthesized as a synthetic DNA and cloned into a commercial vector utilizing T7 promoter based vector namely pET21a vector. The SD stretch fragment was released from the synthetic DNA as an NdeI/EcoRI fragment and cloned into pET21a at the same sites. Nucleotide sequence corresponding to the enterokinase cleavage site was incorporated between BamHI and EcoRI sites in the SD repeat.

The additional amino acids at the N terminal region of the SD repeat units may be GST tag, His tag, T 7 tag Trx tag, MBP tag, His-GM tag etc.

For the present example GM tag is used. The tag is small and hence upon expression, the molar ratio of the gene of interest would be highest for a tag which is the smallest in size since the other well known tags are very large in size. GM tag (the C-terminus domain of hGMCSF) was amplified from a full length human GM-CSF synthetic gene using gene specific primers

SEQUENCE ID 1:
Forward primer: 5′ ccg ccg gaa ttc cat atg cac tac aag cag cac tgc cct cca 3′
SEQUENCE ID 2:
Reverse primer: 5′ ccg ccg gaa ttc ttt atc atc atc atc gga tcc gac tgg ctc cca gca gtc 3′

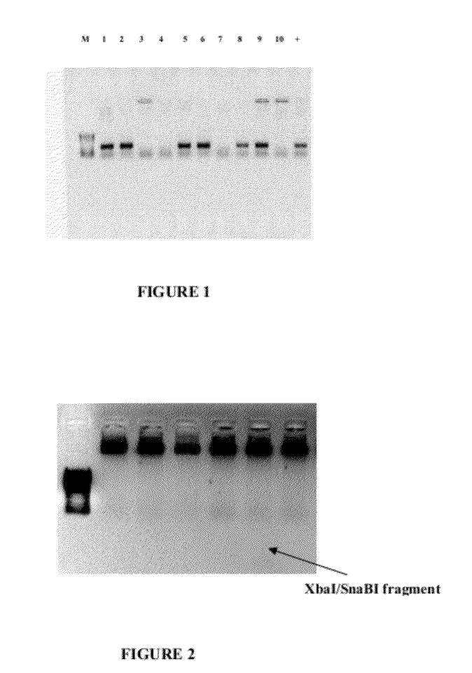

PCR was performed in a total volume of 250 ul containing 100 pg of a synthetic gene (Gene bank accession no. BC108724), 3U of Taq DNA polymerase, 200 uM dNTPs (Bangalore Genei Pvt. Ltd. India) and l Opmoles each of primers (Sigma). Amplification was done in a two step manner at 94° C. for 5 min followed by 5 cycles of 94° C. for 30 s, 50° C. for 30 s and 72° C. for 30 s; 25 cycles of 94° C. for 30 s, 62° C. for 30 s and 72° C. for 30 s and final primer extension at 72° C. for 5 min. The PCR product was digested with NdeI and cloned into pET21a vector (Novagen) as NdeI fragment. The constructed vector was designated as pCGMSD and the enterokinase (EK) cleavage site was introduced into the vector to obtain target protein with no extra amino acids at the N-terminus. Thus the fusion tag vector, pCGMSD was constructed by cloning GM tag and SD repeat into E. coli expression vector pET21a. The incorporation of GM was verified by colony PCR screening with T7 promoter primer and GM reverse primers. FIG. 1 indicates colonies showed PCR product corresponding to GM tag. The incorporation of GM tag was further verified by restriction digestion with XbaI/SnaBI (FIG. 2).

### Example 2

### Cloning of human Granulocyte Colony Stimulating Factor (hGCSF) in pCGMSD

hGCSF was amplified from a synthetic gene using gene specific primers

SEQUENCE ID 3:
Forward: 5′ CCG CCG GGA TCC GAT GAT GAT GAT AAA ACG CCA TTA GGC
CCG GCC 3′
SEQUENCE ID 4:
Reverse: 5′ CCG CCG GAA TTC AAG CCT TAA CGG CTC CGC TAA ATG ACG 3′.

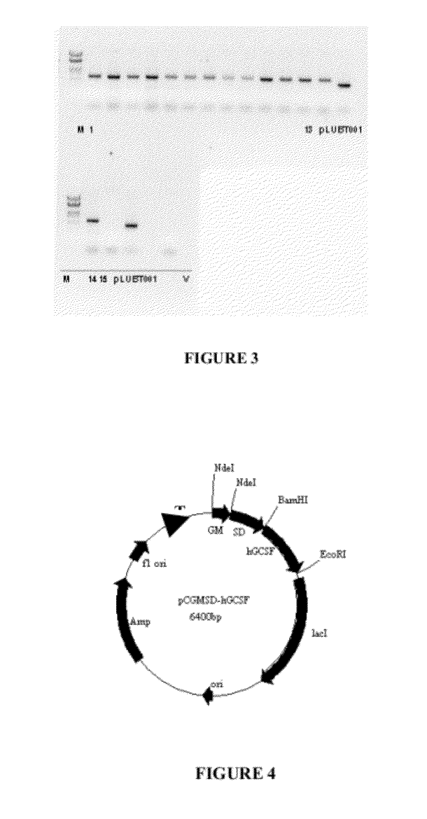

PCR was performed in a total volume of 250 ul containing 100 pg of synthetic gene (Gene bank accession no. DQ914891), 3U of Taq DNA polymerase, 200 uM dNTPs and 10 pmoles each of primers. Amplification was done in a two step manner at 94° C. for 5 min followed by 30 cycles of 94° C. for 30 s, 63° C. for 30 s and 72° C. for 30 s and final primer extension at 72° C. for 5 min. The PCR product was digested with BamHI/EcoRI and cloned into pCGMSD as BamHI/EcoRI fragment. Clones were screened by colony PCR (FIG. 3) and the construct was designated by pCGMSD-hGCSF (FIG. 4).

### Example 3

### Expression of GMSD-hGCSF Fusion Protein in E. coli host BL21(DE3)

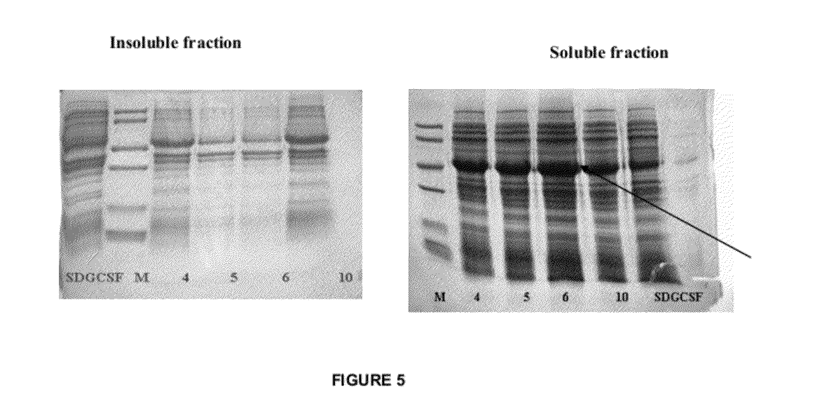

The pCGMSD-hGCSF construct was introduced into E. coli expression host BL21 (DE3) by a method known as transformation. The cells were induced with 1 mM IPTG and induction was carried out for 4 hours as described before. The sub cellular fractionation was done after cell lysis and soluble and insoluble fractions were separated, analysed on SDS-PAGE. FIG. 5 shows more than 80% hGCSF protein was residing in the soluble fraction indicating that GMSD fusion indeed offers solubility to hGCSF. Introduction of enterokinase cleavage site between fusion tag and target protein helps in obtaining target proteins with no extra amino acid at its amino terminus.

### Example 4

### Immunoblot with Anti-hGMCSF Antibody and Purification of GMSD Tag Fusion Proteins

GM fusion proteins could be detected and quantified by immunoblot or ELISA with commercially available anti-hGMCSF antibody. Human GCSF was cloned in pCGMSD vector and expressed in BL21(DE3) E. coli host. Immunoblot analysis was carried out with both mouse anti-hGCSF and rabbit anti-hGMCSF antibodies. GM-GCSF fusion protein is detected by both GCSF as well as GMCSF antibodies. As expected, untagged GCSF is detected only by GCSF antibody and not by GMCSF antibody.

The fusion tag has an affinity to bind to heparin [Sebollela et. al., Journal of Biological Chemistry 280 31049-31956; 2005] and thus can be purified by affinity chromatography using immobilized heparin sepharose matrices. Human IL11 expressed as GM fusion, was allowed to bind to heparin sepharose affinity column at pH 5 and eluted at alkaline pH with buffer containing high salt, IL 11 was found to be purified and fully biologically active.

### Example 5

### Biological Activity of the Fusion Protein

NFS60 cell proliferation assay was carried out to check the biological activity of hGCSF with fusion tag and it has been found to be active in tagged protein.

### Example 6

### Construction of a Fusion Tag Vector and Cloning of Human Tissue Plasminogen Activator (Reteplase), Interleukin-2, Enterokinase and Interleukin-11 Genes in pCGMSD Vector

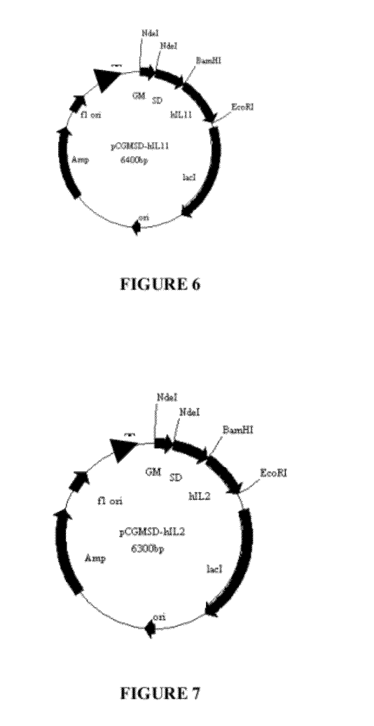

All the above gene products have been reported to occur as insoluble inclusion bodies in E. coli system. All these genes were cloned as BamH1/HindIII into a vector containing GM-SD tag under pET21a vector (FIGS. 6, 7, 8).

All the genes were screened using gene specific PCR and then clones were screened for expression for fusion proteins of GM-SD-Reteplase, GM-SD-IL-2, GM-SD-IL-11 and GM-SD-enterokinase (EK) in BL21(DE3) cells using 1 mM IPTG as the inducer.

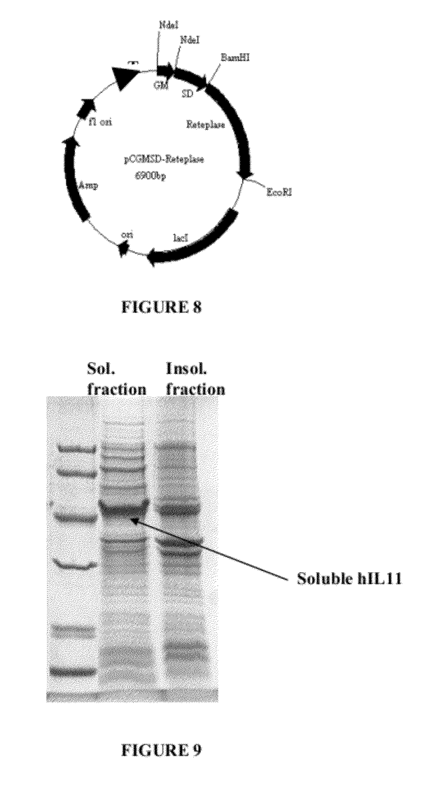

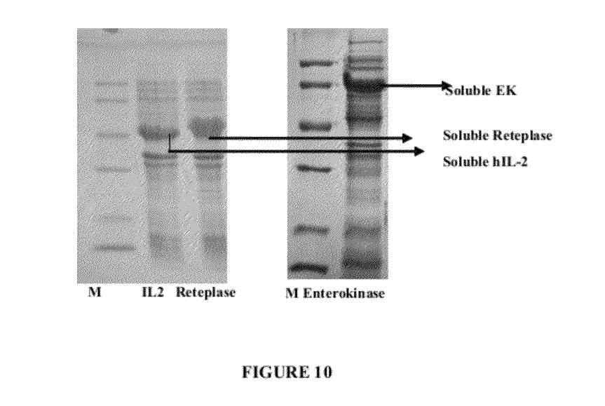

The results indicate expression of the fusion proteins as soluble entities as evident from FIGS. 9 and 10.

### INDUSTRIAL APPLICABILITY

Improved solubility (S)—Fusion of the N-terminus of the target protein to the C-terminus of a soluble fusion partner often improves the solubility of the target protein.
Improved detection (D)—Fusion of the target protein to either terminus of a short peptide (epitope tag) or protein which is recognized by an antibody (Western blot analysis) or by biophysical methods (e.g. GFP by fluorescence) facilitates the detection of the resulting protein during expression or purification.
Improved purification (P)—Simple purification schemes have been developed for proteins used at either terminus which bind specifically to affinity resins.
Localization (L)—Tag, usually located on N-terminus of the target protein, which acts as address for sending protein to a specific cellular compartment.
Improved Expression (E)—Fusion of the N-terminus of the target protein to the C-terminus of a highly expressed fusion partner results in high-level expression of the target protein.
The tag provide for fusion to a polypeptide that itself is highly soluble (e.g. GST, Trx, NusA)
provide for fusion to an enzyme that catalyzes disulfide bond formation (e.g. thioredoxin, DsbA, DsbC)
provide a signal sequence for translocation into the periplasmic space
Proteins, which are prone to insoluble aggregates due to higher content of cysteines, could be easily made soluble using this novel fusion tag.
This provides a cost effective and time saving way of preparation of soluble proteins
This tag could also prove useful for several eukaryotic proteins, which are prone to go to inclusion bodies in E. coli.

1. A fusion tag comprising Serine-aspartic acid (SD) repeat region of Staphylococcus aureus SdrC gene superfamily with a START codon and an enterokinase cleavage site.

2. The fusion tag as claimed in claim 1, comprising 107 amino acids of Serine-aspartic acid repeat region of Staphylococcus aureus SdrC gene superfamily.

3. The fusion tag as claimed in claim 1, comprising nucleotide sequence ID 7
ATGAGCGATTCCGATTCAGACTCGGACTCGGATTCCGATTCCGACAGTGA
TTCAGATTCTGACTCAGATTCCGATTCTGATTCTGATTCGGATTCCGACTC
CGATAGCGACTCAGATAGTGACTCTGACTCGGACAGCGATTCTGATAGCG
ACTCTGATTCCGATAGCGATAGCGATTCAGATAGCGATTCTGACTCGGAT
TCTGATTCCGATTCTGACTCTGACAGCGATTCCGATAGCGACAGCGACTCT
GATAGTGATTCAGACTCTGATTCTGATAGTGATAGCGATTCGGATAGTGG
ATCCGATGATGATGATAAA

4. The fusion tag as claimed in claim 1, comprising amino acid sequence ID 8 as under:
MSDSDSDSDSDSDSDSDSDSDSDSDSDSDSDSDSDSDSDSDSDSDSDSDSD
SDSDSDSDSDSDSDSDSDSDSDSDSDSDSDSDSDSDSDSDSDSDSDSDSGS
DDDDK
wherein D D D D K is the enterokinase cleavage site at the carboxy end of the construct.

5. The fusion tag as claimed in claim 1, further comprising additional amino acid selected from T7tag, GST tag, His tag, Trx tag, MBP tag, GM tag, His-GM tag.

6. The fusion tag as claimed in claim 5, wherein the additional amino acid which is GM tag.

7. A fusion tag comprising Serine-aspartic acid repeat region of Staphylococcus aureus SdrC gene superfamily with a START codon and an enterokinase cleavage site adapted to increase the solubility of proteins.

8. The fusion tag as claimed in claim 7, further comprising additional amino acid selected from T7tag, GST tag, His tag, Trx tag, MBP tag, GM tag, His-GM tag.

9. The fusion tag as claimed in claim 8, wherein the additional amino acid is GM tag.

10. A vector comprising fusion tag having a Serine-aspartic acid repeat region of Staphylococcus aureus SdrC gene superfamily with a START codon and an enterokinase cleavage site.

11. The vector as claimed in claim 10, wherein the fusion tag further comprise of additional amino acid selected from T7tag, GST tag, His tag, Trx tag, MBP tag, GM tag, His-GM tag.

12. The vector as claimed in claim 11, wherein additional amino acid in the fusion tag is GM tag.

13. A kit for expression of soluble proteins comprising vector comprising a fusion tag comprising Serine-aspartic acid repeat region of Staphylococcus aureus SdrC gene superfamily with a START codon and an enterokinase cleavage site and addition amino acids.

14. The kit as claimed in claim 13, wherein the fusion tag further comprise of additional amino acid selected from T7tag, GST tag, His tag, Trx tag, MBP tag, GM tag, His-GM tag.

15. The kit as claimed in claim 14, wherein additional amino acid in the fusion tag is GM tag.

16. A method for producing soluble and active recombinant protein comprising: (a) cloning fusion tag comprising SD repeats in the vector (b) cloning additional amino acid sequence in step (a) (c) introduction of gene of interest in step (b) (d) Transformation of vector from step (d) in E Coli (e) expression of fusion protein (f) Separation of protein of interest from fusion protein.

17. The method as claimed in claim 16, wherein SD repeats has the ability of improving the solubility of protein of interest.

18. The method as claimed in claim 16, wherein the fusion tag further comprises additional amino acid selected from T7tag, GST tag, His tag, Trx tag, MBP tag, GM tag, His-GM tag.

19. The method as claimed in claim 18, wherein additional amino acid in the fusion tag is GM tag.
